SEQUENCE LISTING (3 sequences)

SEQ ID NO: 1 - protein, 17 aa, Homo sapiens
GTQNPSQDQGNSPSGKG

SEQ ID NO: 2 - protein, 51 aa, Homo sapiens
NKQEGRDHDKSKGHFHRVVIHHKGGKAHRGTQNPSQDQGNSPSGKGISSQY

SEQ ID NO: 3 - protein, 51 aa, Homo sapiens
YKQEGRDHDKSKGHFHMIVIHHKGGQAHHGTQNPSQDQGNSPSGKGLSSQC

(19) United States
(12) Patent Application Publication
(10) Pub. No.: US 2010/0151498 A1
(43) Pub. Date: Jun. 17, 2010

(54) Method of Diagnosing Asthenozoospermia

(76) Inventors: Teruaki Iwamoto, Kanagawa (JP); Miki Yoshiike, Kanagawa (JP); Kaoru Yoshida, Kanagawa (JP); Kazutaka Terai, Tokyo (JP)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/595,139

(22) PCT Filed: Feb. 19, 2008
(86) PCT No.: PCT/JP2008/052703

§ 371 Date: Dec. 31, 2009

(30) Foreign Application Priority Data
Apr. 13, 2007 (JP) 2007-105708

(51) Int. Cl. G01N 33/567

(52) U.S. Cl. 435/7.21

(57) ABSTRACT

After sampling semen from healthy males and from patients whose chief complaint was infertility, the semen volume, sperm concentration, sperm motility rate, and sperm survival rate in each of the specimens were measured, sperm-bound SPMI was detected using an anti-SPMI antibody, and the amount and rate of SPMI binding were calculated. As a result, only patient specimens were found in the specimen group with amount and rate of SPMI binding above a certain level. Diagnosis of asthenozoospermia can be made by determining the amount and rate of SPMI binding.

### TECHNICAL FIELD

The present invention relates to novel methods for diagnosing asthenozoospermia.

### BACKGROUND ART

Asthenozoospermia is an example of male infertility in which sperm motility is reduced. In the diagnosis of asthenozoospermia, generally semen sperm are observed under a microscope, and the percentage of sperm with normal mobility (motility rate) is determined. However, this method leaves room for the observer's subjectivity in the evaluation of mobility, and the evaluation may differ between different observers or even with the same observer, and therefore it cannot be considered a very highly reliable method. In addition, for male infertility patients, the processing time from semen sampling to examination may have a major effect on the results, thus making consigned examination outside an institution difficult.

Semenogelin (Sg) is a protein secreted by the seminal vesicles, and it is mixed with sperm and prostatic fluid at the time of semen ejaculation. The known human semenogelins are 52 kD semenogelin I (SgI) and 71 kD or 76 kD (glycosylated) semenogelin II (SgII), and SgI and SgII complex to form the main structure of semen coagulum. Semen becomes liquefied several minutes after ejaculation, and the liquefaction is believed to occur as a result of decomposition and low molecularization of semenogelin by prostate specific antigen (PSA).

Seminal plasma motility inhibitor (SPMI) is a PSA-decomposed fragment of semenogelin. SPMI has the activity to control sperm motility (Non-patent document 1), and is thought to be a factor in asthenozoospermia. While proteins and ions that affect sperm motility have been reported, no other factor has been clearly linked to asthenozoospermia in a cause-and-effect relationship. In regard to the relationship between SPMI and asthenozoospermia, the present inventors have already reported that SPMI binds to defective sites in the membrane structure of the sperm of asthenozoospermia patients, and that while a weak correlation is seen between SPMI concentration in seminal fluid and sperm motility rate, no significant difference was found in the average SPMI concentration between the poor motility sperm group and good motility sperm group in healthy males (Non-patent document 2).

Since SPMI is specifically found in seminal fluid, methods have been developed for identifying the presence of semen in forensic medicine samples by SPMI detection (Patent documents 1 and 2). However, though it has attracted attention as a factor in asthenozoospermia, no report has yet linked SPMI detection with asthenozoospermia diagnosis.

[Patent document 1] Japanese Patent Application Kokai Publication No. (JP-A) 2000-283982 (unexamined, published Japanese patent application)
[Patent document 2] JP-A (Kokai) 2004-2279
[Non-patent document 1] J. Androl. 1988; 9:377-383
[Non-patent document 2] J. Androl. 2003 November-December; 24(6):878-84

### DISCLOSURE OF THE INVENTION

### Problems to be Solved by the Invention

The present invention has been accomplished in light of the circumstances described above, and it is an objective of the invention to provide novel methods for diagnosing asthenozoospermia.

### Means for Solving the Problems

With an aim to solve the aforementioned problems, the present inventors have conducted diligent research. They collected semen samples from healthy males and patients complaining chiefly of infertility, and for each of the specimens, measured semen volume, sperm concentration, sperm motility rate, and sperm survival rate. They also detected sperm-bound SPMI using an anti-SPMI antibody, and calculated the amount and rate of SPMI binding. As a result, it was found that the group of specimens with an amount and rate of SPMI binding above a certain level contain only patient-derived specimens. Until now, SPMI has been considered to be a factor in asthenozoospermia, but reported results have suggested that it is difficult to diagnose asthenozoospermia based on detection of SPMI in seminal fluid. The present inventors' current findings are a result of changing the viewpoint and focusing on a factor in sperm that is believed to be under the action of SPMI. The present invention, therefore, relates to methods for diagnosing asthenozoospermia based on the amount and rate of SPMI binding, and specifically provides the following inventions:

(1) a method for diagnosing asthenozoospermia comprising a step of detecting seminal plasma motility inhibitor (SPMI) bound to sperm in a specimen taken from a subject, and determining the amount and rate of SPMI binding;
(2) a method for detecting reduction in the quality of sperm, comprising a step of detecting seminal plasma motility inhibitor bound to sperm in a specimen taken from a subject, and determining the amount and rate of SPMI binding;
(3) the method according to (1) or (2) above, which comprises steps (a) and (b) below:
(a) a step of contacting a specimen taken from a subject with an anti-SPMI antibody or anti-semenogelin antibody, detecting the SPMI bound to the sperm in the specimen, and calculating the amount and rate of SPMI binding, and
(b) a step of determining that the sperm quality of the subject is reduced if the amount and rate of SPMI binding are greater than the amount and rate of SPMI binding for a healthy male;
(4) the method according to (3) above, wherein the anti-SPMI antibody or anti-semenogelin antibody is an antibody that recognizes the amino acid sequence of SEQ ID NO: 1 of SPMI or semenogelin; and
(5) a kit for diagnosis of asthenozoospermia, which comprises an anti-SPMI antibody and a sperm-labeling reagent.

### BRIEF DESCRIPTION OF THE DRAWINGS

FIG. 1 is a graph showing the relationship between sperm motility rate and SPMI binding rate for asthenozoospermia patients and healthy males.

FIG. 2 is a graph showing the relationship between sperm survival rate and SPMI binding rate for asthenozoospermia patients and healthy males.

FIG. 3 is a graph showing the relationship between amount and rate of SPMI binding for asthenozoospermia patients and healthy males.

### BEST MODE FOR CARRYING OUT THE INVENTION

The present invention provides methods for diagnosing asthenozoospermia comprising a step of detecting seminal plasma motility inhibitor (SPMI) bound to sperm in a specimen taken from a subject, and determining the amount and rate of SPMI binding. Asthenozoospermia is an example of male infertility in which sperm motility is reduced. The methods of the present invention allow objective diagnosis of the sperm quality of subjects by detecting binding between sperm in a specimen and SPMI, and determining the amount and rate of SPMI binding.

The present invention further provides methods for examining asthenozoospermia, which comprise detecting sperm-bound seminal plasma motility inhibitor (SPMI) in a specimen taken from a subject, determining the amount and rate of SPMI binding, and deciding the condition of the subject based on the obtained data.

SPMI (14 kDa) is a PSA-decomposed product of semenogelin. The amino acid sequences of SPMI are shown below.

Sg-I derived SPMI:

(SEQ ID NO: 2)

NKQEGRDHDKSKGHFHRVVIHHKGGKAHRGTQNPSQDQGNSPSGKGISS
QY
Sg-II derived SPMI:

(SEQ ID NO: 3)

YKQEGRDHDKSKGHFHMIVIHHKGGQAHHGTQNPSQDQGNSPSGKGLSS
QC

Semenogelin is a protein secreted by seminal vesicles, and semenogelin I (462 amino acid residues, 52 kDa) and semenogelin II (582 amino acid residues, 71 kDa or 76 kDa in glycosylated form) exist in humans. The gene sequence and amino acid sequence of semenogelin are known, and are registered in the database as semenogelin I: Accession No. BC007096 and semenogelin II: Accession No. M81652. The amino acid sequences of semenogelin I and semenogelin II have approximately 80% homology. The SPMI of the present invention may be a decomposition product of either semenogelin I or II. SPMI corresponds to amino acids 85 to 136 of the amino acid sequence of semenogelin I or II.

A specimen according to the present invention is semen taken from a subject, or washed sperm obtained by separating seminal fluid from the semen. The animal species from which the specimen is derived is a primate, including a human.

Sperm-bound SPMI in the specimen may be detected using an anti-SPMI antibody by means known to a person having ordinary skill in the art. For instance, as described in the Examples, washed sperm from the semen of a subject may be prepared as specimen, and the number of sperm and SPMI-bound sperm and the amount of sperm-bound SPMI can be determined by adding an anti-SPMI antibody and a labeled secondary antibody to the specimen, labeling the sperm with a nuclear staining agent and the like, and detecting the signal emitted from each labeling substance by flow cytometry.

The anti-SPMI antibody used in the methods of the present invention may be a polyclonal antibody or monoclonal antibody, so long as it is an antibody that specifically recognizes SPMI. The phrase “specifically recognizes” means to exclude antibodies that bind to SPMI nonspecifically. The anti-SPMI antibodies of the present application recognize a portion of semenogelin I or II in SPMI and bind to semenogelin I or II, but do not recognize or bind to SPMI in a nonspecific manner. The anti-SPMI antibody used in the methods of the present invention is preferably an antibody that recognizes both the SPMI that is a decomposition product of semenogelin I and the SPMI that is a decomposition product of semenogelin II, or in other words, an antibody that recognizes a sequence common to semenogelin I and semenogelin II. A sequence common to semenogelin I and II can be easily found by a person of ordinary skill in the art by comparing the known amino acid sequences of semenogelin I and semenogelin II. An example of such common sequence is

| | |
|---|---|
| GTQNPSQDQGNSPSGKG. | (SEQ ID NO: 1) |

The anti-SPMI antibody used in the methods of the present invention may be produced by procedures well known to those skilled in the art. Specifically, a polyclonal anti-SPMI antibody can be prepared by immunizing an animal by injection with an immunogen and if necessary an immunoadjuvant, sampling blood from the immunized animal and confirming the increase in antibody titer, and then bleeding the immunized animal to obtain antiserum. Also, in the case of an anti-monoclonal antibody, animals may be immunized and hybridomas are created by fusing myeloma cells with B cells obtained from the spleens extracted from animals with increased antibody titer, and an anti-SPMI antibody is prepared from culture supernatants of antibody-producing cells screened from the hybridomas. The immunogen may be naturally occurring SPMI prepared from liquefied semen, or a partial polypeptide thereof. The full-length or partial SPMI may also be artificially synthesized. For example, an SPMI fragment comprising the amino acid sequence of SEQ ID NO: 1 may be used as the immunogen. Alternatively, the entire semenogelin I or II or a semenogelin fragment comprising a sequence common to SPMI may be used as the immunogen to produce antibody that recognizes SPMI. The type of animal immunized is not particularly restricted. Examples include mouse, rat, guinea pig, hamster, rabbit, goat, donkey, horse, pig, sheep, cow, dog, monkey and other animals. Human antibody may also be used for the methods of the present invention. Examples of preparing an anti-SPMI antibody are described in JP-A (Kokai) 2000-283982, JP-A (Kokai) 2004-2279, and J Androl. 2003 November-December; 24(6):878-84.

Sperm labeling may be carried out using known nuclear stains such as propidium iodide. Alternatively, an antibody known as anti-sperm antibody or an antibody against a specific surface antigen on sperm may be labeled for secondary labeling of the sperm.

The “amount of SPMI binding” and “rate of SPMI binding” according to the present application can be determined from the numerical values of the total sperm volume in the specimen, the volume of SPMI-bound sperm, and the amount of sperm-bound SPMI obtained by the procedure described above. According to the present invention, “amount of SPMI binding” means the amount of SPMI bound to sperm. The amount of SPMI binding may be expressed directly as the mass or number of moles of SPMI, or it may be expressed indirectly as a parameter that reflects the amount of SPMI. For example, it may be expressed as the amount of signal of a labeled compound that reflects the amount of SPMI. According to the present invention, “rate of SPMI binding” means the percentage of SPMI-bound sperm in the measured sperm.

The sperm quality of a subject is judged to be reduced if the amount of SPMI binding and rate of SPMI binding in the subject calculated in the manner described above are greater than the amount of SPMI binding and rate of SPMI binding for a healthy male. According to the present invention, “sperm quality” means quality of the sperm's function in fertilization.

In the Examples, healthy persons with a sperm motility rate of 50% or greater were found to be limited to amount of SPMI binding below a certain level and rate of SPMI binding below a certain level. According to WHO standards, cases of less than 50% sperm motility rate are defined as asthenozoospermia. Therefore, in the methods of the present invention, it is appropriate to suspect asthenozoospermia when a case demonstrates an amount of SPMI binding or rate of SPMI binding above a certain level.

“Amount of SPMI binding above a certain level” means, for example, 120 or greater and preferably 450 or greater.

“Rate of SPMI binding above a certain level” means, for example, 42% or greater and preferably 55% or greater.

The anti-SPMI antibody of the present invention may be combined with a sperm-labeling reagent to constitute a kit for diagnosis of asthenozoospermia. A kit for asthenozoospermia diagnosis according to the present invention may also comprise other reagents as necessary, for example, a solution for preparing washed sperm. The constitution of a diagnosis kit according to the present invention may be suitably changed according to the user, and for example, it may include reagents for professionals such as doctors or laboratory technicians, as well as reagents that incorporate tools for convenient operation for home use.

All of the prior art documents cited throughout the present specification are incorporated herein by reference.

### EXAMPLE

The present invention will now be explained in greater detail by the following examples; however, the present invention is not limited by these examples.

### Example

In order to examine whether SPMI binding to sperm can be used as a diagnostic parameter for asthenozoospermia, sperm-SPMI binding in semen was analyzed by flow cytometry using an anti-human SPMI antibody. This example was carried out with approval of the Ethics Committee, St. Marianna University School of Medicine.

### (Method)

Eleven specimens of semen from 5 healthy males and 18 specimens of semen from 16 patients with sperm motility rate of less than 50%, who visited the hospital between June and August of 2006 with chief complaint of infertility, were obtained. The sufficiently liquefied semen specimens were centrifuged with 65% Percoll to prepare washed sperm. The liquid volume, sperm concentration, motility rate, and survival rate of each specimen were measured before and after washing.

Binding of sperm with SPMI was analyzed in the following manner. The washed sperm were fixed with 2% PFA and reacted with an anti-human SPMI monoclonal antibody (F11), and then with Alexa488-labeled anti-mouse IgG. After nuclear staining with propidium iodide (PI), the binding of SPMI with sperm was detected by flow cytometry, and the following were obtained:

Rate of SPMI binding(%)=(number of SPMI-bound sperm in the specimen/number of sperm measured)×100

Amount of SPMI binding (mean Alexa488 fluorescence intensity of SPMI-bound sperm)

### (Results)

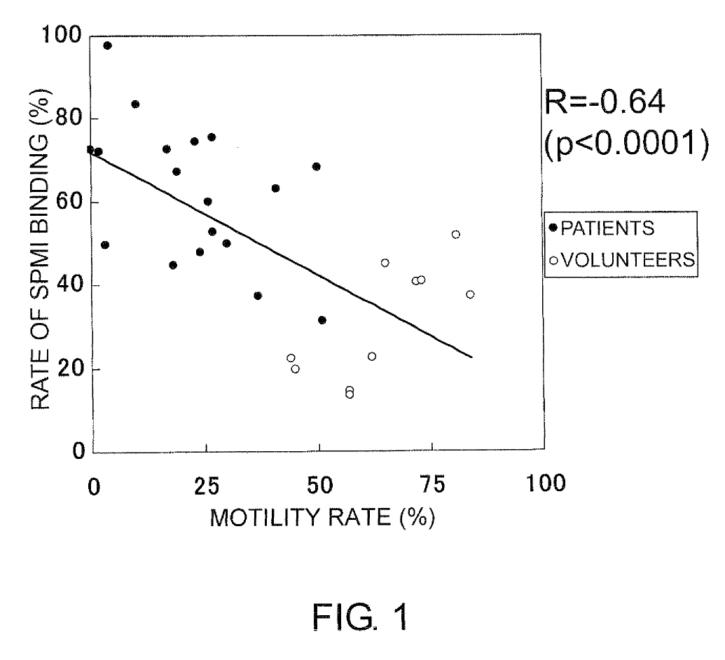

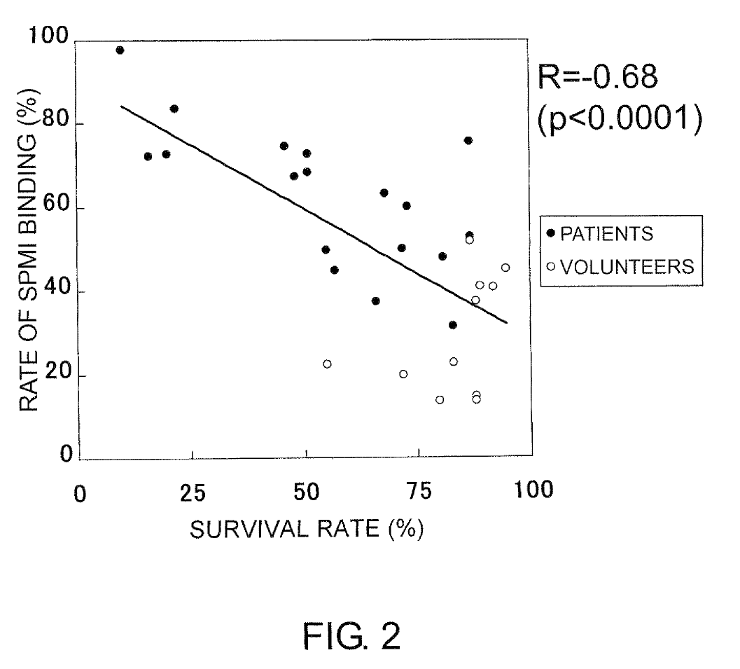

The rates of SPMI binding (mean±SD) were 63±17 (%) for patients and 29±14 (%) for healthy males, confirming a significantly higher rate of SPMI binding for the patients than for the healthy males (p<0.0001). A negative correlation was found between SPMI binding rate and sperm motility rate (FIG. 1, r=−0.64, p<0.0001), and a negative correlation was also found between SPMI binding rate and sperm survival rate (FIG. 2, r=−0.68, p<0.0001).

Similar to the rate of SPMI binding, the amount of SPMI binding (mean±SD) was significantly higher for patients (579±456) than for healthy males (184±143). A negative correlation was found between the amount of SPMI binding and sperm motility rate (r=−0.37, p=0.046), and a negative correlation was also found between the amount of SPMI binding and sperm motility rate (r=−0.45, p=0.013).

A positive correlation was found between the rate of SPMI binding and amount of SPMI binding (r=0.538, p=0.002).

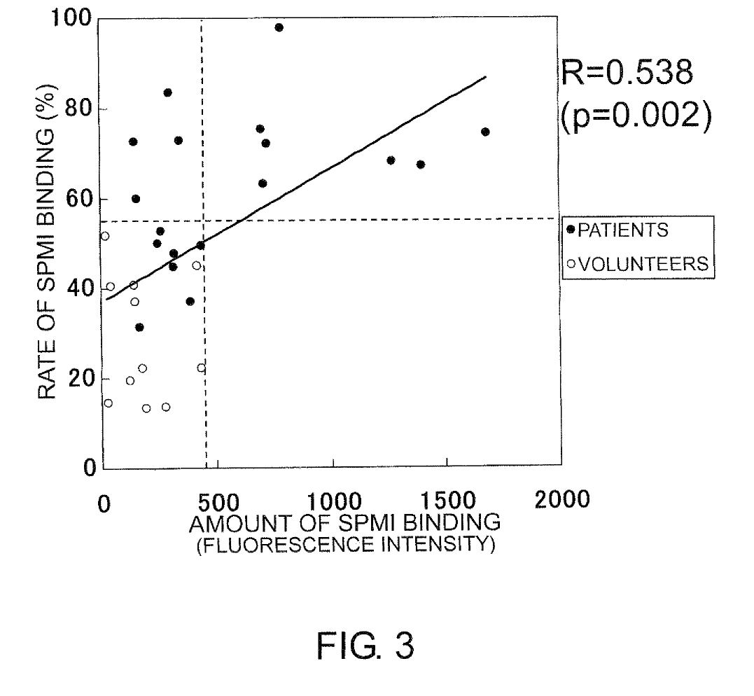

Thus, the amount of SPMI binding was greater in specimens with high SPMI binding rates, and this tendency was significant only in the patient group. As shown in FIG. 3, only patients were found to be distributed at SPMI binding rate of 55% or more or binding amount of 450 or more, and therefore these numerical values were thought to be the cutoff values for healthy persons in the groups studied here. Patients with specimens exhibiting values at or above the cutoff values had SPMI binding to sperm and reduced sperm motility, and were conjectured to be exhibiting infertility symptoms. These results indicated that detection of SPMI binding to sperm is useful as a diagnostic parameter for asthenozoospermia.

### INDUSTRIAL APPLICABILITY

The present invention provides novel methods for diagnosing asthenozoospermia. Based on detection of binding between sperm and SPMI, the methods of the present invention determine not only sperm motility but also quality, including fertilization capacity. Thus, the methods of the present invention allow objective diagnosis without subjective influence of the observer which is a factor in conventional methods. As an additional advantage, the method is not affected by the time of specimen sampling to measurement, and thus, it is possible for private clinics that lack measuring equipment to outsource examination to consigned companies. It is thereby possible to provide appropriate treatment for infertility patients early.

1. A method for diagnosing asthenozoospermia comprising a step of detecting seminal plasma motility inhibitor (SPMI) bound to sperm in a specimen taken from a subject, and determining the amount and rate of SPMI binding.

2. A method for detecting reduction in the quality of sperm, comprising a step of detecting seminal plasma motility inhibitor bound to sperm in a specimen taken from a subject, and determining the amount and rate of SPMI binding.

3. A method for examining asthenozoospermia, which comprises detecting seminal plasma motility inhibitor (SPMI) bound to sperm in a specimen taken from a subject and determining the amount and rate of SPMI binding, and determining the condition of the subject based on the obtained data.

4. The method according to any one of claims 1 to 3, which comprises steps (a) and (b) below:
(a) a step of contacting a specimen taken from a subject with an anti-SPMI antibody or anti-semenogelin antibody, detecting the SPMI bound to the sperm in the specimen, and calculating the amount and rate of SPMI; and
(b) a step of determining that the sperm quality of the subject is reduced if the amount and rate of SPMI binding are greater than the amount and rate of SPMI binding for a healthy male.

5. The method according to claim 4, wherein the anti-SPMI antibody or anti-semenogelin antibody is an antibody that recognizes the amino acid sequence of SEQ ID NO: 1 of SPMI or semenogelin.

6. A kit for diagnosis of asthenozoospermia, which comprises an anti-SPMI antibody and a sperm-labeling reagent.
